(19) United States
(12) Patent Application Publication
(10) Pub. No.: US 2026/0083914 A1
(43) Pub. Date: Mar. 26, 2026

(54) A medicament delivery device and a subassembly of a medicament delivery device

(71) Applicant: SHL Medical AG, Zug (CH)

(72) Inventors: Daniel Säll, Nacka Strand (SE); Tai-Fu Wang, Taoyuan City (TW); Chun-Hsien Huang, Taoyuan City (TW)

(21) Appl. No.: 19/110,476

(22) PCT Filed: Aug. 29, 2023
(86) PCT No.: PCT/EP2023/073651

§ 371 Date: Mar. 11, 2025

Related U.S. Application Data
(60) Provisional application No. 63/430,739, filed on Dec. 7, 2022.

(30) Foreign Application Priority Data
Sep. 13, 2022 (EP) 22195481.1

(51) Int. Cl. A61M 5/20; A61M 5/315; A61M 5/32

(52) U.S. Cl. CPC A61M 5/20; A61M 5/31566; A61M 5/31586; A61M 5/3202; A61M 2005/2026; A61M 2205/332; A61M 2205/44; A61M 2205/502; A61M 2205/581

(57) ABSTRACT

The present disclosure generally relates to medicament delivery devices such as autoinjectors, and particularly concerns a subassembly of a medicament delivery device.

### TECHNICAL FIELD

The present disclosure generally relates to medicament delivery devices such as autoinjectors, and particularly concerns a subassembly of a medicament delivery device.

### BACKGROUND

A number of medical conditions require injections. These days, a number of different injection devices exist, including various types of pen injectors, autoinjectors and on-body devices. Although many of these devices have enabled major improvements in the management of a number of medical conditions, various limitations do still exist in the current technology. Not least amongst these are the difficulties faced by patients that require frequent injections and by patients that need to inject particularly viscous drugs. In considering these problems, the applicant has appreciated that various developments could be made to help improve the medicament delivery devices on the market today, for example concerning manual operation of the outer cap of the medicament delivery devices, which are set out in more detail below.

### SUMMARY

An object of the present disclosure is to provide a subassembly of a medicament delivery device, and a medicament delivery device which solves, or at least mitigates problems of the prior art.

According to a first aspect of the present disclosure, there is provided a subassembly of a medicament delivery device configured to expel medicament from a medicament container having a proximal membrane, the subassembly comprising: a retainer having a distal tubular portion and a proximal tubular portion; a needle hub comprising a needle having a proximal end and an opposite distal end, the needle hub extending from a proximal portion to a distal portion; a proximal housing part having a proximal end portion defining a proximal opening through which the needle extends in a proximal direction during a medicament delivery operation; an outer cap operably attached to the proximal end portion of the proximal housing part, wherein the outer cap is operably connected to the needle hub such that when the outer cap is manually operated, the needle hub is distally and coaxially moved within the proximal tubular portion of the retainer whereby the distal end of the needle penetrates the proximal membrane; a supplemental device attached to the outer cap, the supplemental device comprising a battery, an activation member and a communication module configured to transmit device status data to an external device upon activation of the activation member, wherein the activation member is configured to assume a non-activated state when being arranged in a fixed position relative to the proximal end portion of the proximal housing part; wherein the activation member is configured to move from its non-activated state into an activated state when being moved from the fixed position relative to the proximal end portion when the outer cap is moved relative to the proximal housing part.

Embodiments of the present disclosure advantageously provides for activation of the activation member when the outer cap is moved relative to the proximal housing part. In this way, the communication module may transmit device status data to an external device informing the latter of that a patient is using the medicament delivery device.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the components thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, “circumferentially” refer to a circumference or a circumferential direction 131 relative to an axis 112, typically a central axis extending in the direction of the longest extension of the device and/or component. Similarly, “radial” or “radially” refer to a direction 132 extending radially relative to the axis, and “rotation”, “rotational”and “rotationally”refer to rotation relative to the axis.

According to one embodiment, the communication module is configured to transmit device status data upon activation of the activation member. This provides for an activation of the communication module. Preferably, the activation member is electrically and/or communicatively connected to the communication module, e.g. by means of a PCB board.

According to one embodiment, the proximal end portion of the proximal housing part comprises a proximal end surface, wherein the fixed position of the activation member is defined by that the activation member abuts the proximal end surface. Hereby, the non-activated state of the activation member is defined in relation to proximal end portion of the proximal housing part in a straightforward manner.

According to one embodiment, the activation member is configured to move to its activated state when the outer cap is manually operated to be removed from the proximal housing part. That is, the outer cap may be manually operated by the act of cap removal. This is advantageous as the communication module transmits device status data upon cap removal and may thereby inform the external device that a patient has removed the outer cap in order to use the medicament delivery device. As the supplement device is attached to the outer cap, the activation member and the outer cap are typically configured to be moved together.

According to one embodiment, the device status data transmitted from the communication module upon activation of the activation member comprises information of that the needle has penetrated the proximal membrane of the medicament container and the outer cap is distant relative to the proximal housing part. This is advantageous as the communication module transmits device status data upon cap removal with information of that the needle has penetrated the proximal membrane of the medicament container, and may thereby inform the external device that the medicament delivery device is ready for use.

According to one embodiment, the subassembly further comprises a cap clutch proximally attached to the proximal end portion of the proximal housing part, the cap clutch comprising a socket with at least one activation ramp, wherein the fixed position of the activation member is defined by that the activation member is arranged within the socket at a fixed axial distance from the proximal end portion. Hereby, the non-activated state of the activation member is defined in relation to the socket in a straightforward manner.

According to one embodiment, the activation member is configured to move to its activated state by moving the activation member out of the socket via the activation ramp, when the outer cap is rotationally moved relative to the proximal housing part. This is advantageous as the communication module transmits device status data upon rotation of the outer cap and may thereby inform the external device that a patient is rotating the outer cap in order to use the medicament delivery device. As the supplement device is attached to the outer cap, the activation member and the outer cap are typically configured to be rotated together.

According to one embodiment, the outer cap comprises a distal portion with an inner surface comprising axially and radially extending protrusions, wherein the cap clutch comprises at least one radially flexibly arranged tongue arranged to interact with the axially and radially extending protrusions by a ratchet mechanism, such that the radially flexibly arranged tongue engages with the axially radially extending protrusions to rotate the cap clutch when the outer cap is being rotated in a first rotational direction, and such that the radially flexibly arranged tongue slides up and over the axially and radially extending protrusions to generate a click sound when the outer cap is being rotated in an opposite direction to the first rotational direction. By the ratchet mechanism, the cap clutch is only rotated upon a rotation of the outer cap in the first rotational direction. When rotating the outer cap in the opposite direction to the first rotational direction, the cap clutch is not rotated.

According to one embodiment, the supplemental device further comprises a sensor activable by the activation member and configured to identify the click sounds generated by the ratchet mechanism when the outer cap is being rotated in the opposite direction to the first rotational direction. This is advantageous as the communication module thereby may transmit device status data comprising information of the rotational direction of the outer cap and may thereby inform the external device that a patient is rotating the outer cap without rotating the cap clutch.

According to one embodiment, the sensor is an accelerometer. This advantageously provides for a reliable detection of the click sounds generated by the ratchet mechanism when the outer cap is being rotated in the opposite direction to the first rotational direction.

According to one embodiment, the device status data transmitted from the communication module upon activation of the activation member comprises information of the rotational direction of the outer cap based on the presence and absence of identification of click sounds by the sensor. This is advantageous as the communication module thereby may transmit device status data comprising information of the rotational direction of the outer cap regardless of if the outer cap is rotated in the first rotational direction or opposite the first rotational direction.

According to one embodiment, the device status data transmitted from the communication module upon activation of the activation member comprises information that the rotational direction of the outer cap is wrong in response to identification of click sounds by the sensor. Thus, rotation of the outer cap in a rotational direction opposite to the first rotational direction may be referred to as the “wrong” rotational direction, while rotation of the outer cap in the first rotational direction may be referred to as the “correct” rotational direction.

According to one embodiment, the subassembly further comprising a light-emitting source, wherein the communication module is configured to activate the light-emitting source in response to receiving information that the rotational direction of the outer cap is wrong. This is advantageous as the patient receives a direct and visual feedback that the rotation of the outer cap is performed in the wrong rotational direction.

According to one embodiment, the activation member is retractable, and is arranged retracted in its non-activated state, and is arranged protracted in its activated state. This provides for a reliable movement of the activation member from its non-activated state to its activated state.

According to one embodiment, the activation member is an electronic switch. This provides for a reliable activation of the activation member when being moved from its non-activated state to its activated state. Moreover, an electronic switch may communicate with the communication module in a reliable manner.

Alternatively, or additionally, according to one embodiment, the activation member comprises a first conductive end and a second conductive end. The first conductive end is operably connected to the battery, and the second conductive end is operably connected to the communication module. In this example, the proximal housing part comprises an insulating sheet. When the outer cap is adjacent to the proximal housing part, the insulating sheet is positioned between the first conductive end and the second conductive end such that the activation member is in non-activated state as the electrical connection between the battery and the communication module is ruptured by the insulating sheet. Once the outer cap is moved away from the proximal housing part, activation member is moved away from the insulating sheet such that the first conductive end is connected to the second conductive end. As a result, the electrical connection between the battery and the communication module is established and the activation member is in its activated state.

According to one embodiment, the supplemental device further comprises a memory configured to store the status device data, wherein the communication module is configured to transmit the device status data wirelessly to the external device. Hereby, the memory can at least temporarily store status device data prior to that the communication module wirelessly transmit the device status data to the external device.

According to one embodiment, the proximal tubular portion of the retainer comprises a first engaging structure, wherein the retainer comprises an outer surface comprising a second engaging structure. Hereby, the retainer may engage with other components in the subassembly in an advantageous manner.

According to one embodiment, the subassembly further comprises a needle cap having a first inner surface rotationally locked with the proximal portion of the needle hub, and a second inner surface comprising a third engaging structure engageable with the second engaging structure of the retainer, where the distal portion of the needle hub comprises a fourth engaging structure engageable with the first engaging structure of the proximal tubular portion of the retainer. Hereby, the needle hub and the retainer may be arranged to move relative each other in a reliable manner, as exemplified below.

According to one embodiment, the first engaging structure is an inner thread in the proximal tubular portion, the second engaging structure is an outer thread in the outer surface, the third engaging structure is an inner thread in the second inner surface of the needle cap, and the fourth engaging structure is an outer thread of the distal portion of the needle hub, wherein the outer cap is configured to upon rotation in the first direction, rotate the needle cap such that the internal thread of the needle cap disengages from the outer thread of the retainer to enable the outer cap and the needle cap to be axially moved proximally away from the proximal housing part, and such that the outer thread of the distal portion of the needle hub rotatably engages with the inner thread of the proximal tubular portion of the retainer to distally move the needle hub coaxially within the retainer. Hereby, manual operation of the outer cap results in that the needle hub distally and coaxially moves within the proximal tubular portion of the retainer whereby the distal end of the needle penetrates the proximal membrane.

According to one embodiment, the distal tubular portion of the retainer is configured to house the proximal membrane of the medicament container. This is advantageous as the distal end of the needle can penetrate the proximal membrane as it moves within the distal tubular portion of the retainer.

According to a second aspect of the present disclosure there is provided a medicament delivery device configured to expel medicament from a medicament container. The medicament delivery device comprises a sub-assembly according to the first aspect of the present disclosure.

Further embodiments of, and effects obtained through this second aspect of the present disclosure are largely analogous to those described above for the first aspect of the present disclosure.

Generally, all terms used in the claims are to be interpreted according to their ordinary meaning in the technical field, unless explicitly defined otherwise herein. All references to “a/an/the member, apparatus, component, means, etc.” are to be interpreted openly as referring to at least one instance of the member, apparatus, component, means, etc., unless explicitly stated otherwise.

### BRIEF DESCRIPTION OF THE DRAWINGS

The specific embodiments of the inventive concept will now be described, by way of example, with reference to the accompanying drawings, in which:

FIG. 1 is a perspective view of a medicament delivery device according to embodiments of the present disclosure;

FIG. 2 is an exploded view of a subassembly of the medicament delivery device of FIG. 1 according to embodiments of the present disclosure;

FIG. 3 is a detailed view of the subassembly of FIG. 2 according to embodiments of the present disclosure;

FIG. 4 is a perspective detailed view of a component of the subassembly in FIGS. 2 and 3 according to embodiments of the present disclosure;

FIG. 5 is a perspective detailed view of a yet another component of the subassembly in FIGS. 2 and 3 according to embodiments of the present disclosure;

FIGS. 6 and 7 are perspective detailed views of components of the subassembly in FIGS. 2 and 3 according to embodiments of the present disclosure;

FIG. 8 is a perspective view of an alternative subassembly of the medicament delivery device of FIG. 1 according to embodiments of the present disclosure;

FIGS. 9 and 10 are perspective detailed views of components of the subassembly in FIG. 8 according to embodiments of the present disclosure;

FIG. 11 is a detailed view of components of the subassembly in FIG. 8 according to embodiments of the present disclosure;

FIG. 12 is a perspective detailed view of a component of the subassembly of FIG. 8 according to embodiments of the present disclosure.

FIGS. 13-18 are another embodiment of the invention.

### DETAILED DESCRIPTION

The inventive concept will now be described more fully hereinafter with reference to the accompanying drawings, in which exemplifying embodiments are shown. The inventive concept may, however, be embodied in many different forms and should not be construed as limited to the embodiments set forth herein; rather, these embodiments are provided by way of example so that this disclosure will be thorough and complete, and will fully convey the scope of the inventive concept to those skilled in the art. Like numbers refer to like members throughout the description.

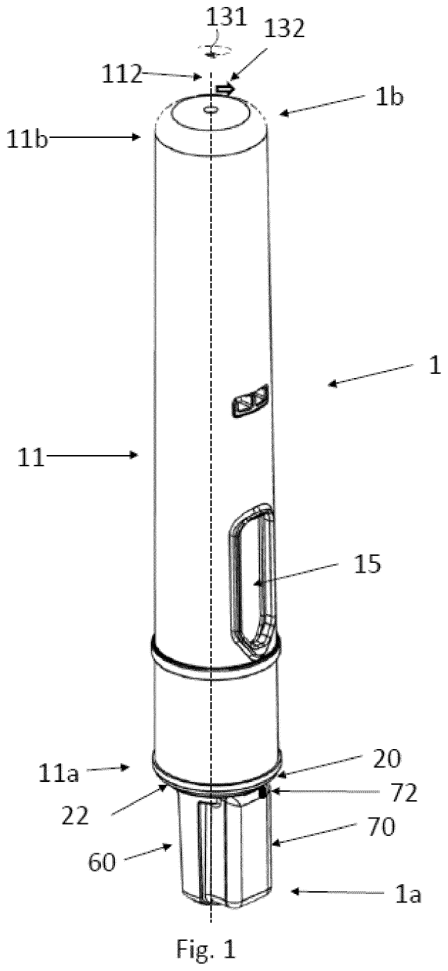

FIG. 1 shows an example of a medicament delivery device 1 such as an autoinjector according to embodiments of the present disclosure. The medicament delivery device 1 is configured to expel medicament from a medicament container 15 via a medicament delivery member such as a needle (not shown in FIG. 1), to a patient at a dose delivery site. The medicament delivery device 1 extends from a proximal end 1a to a distal end 1b relative to the axis 112. The axis 112 is in FIG. 1 a centre axis, from which a circumferential direction 131 relative to the centre axis 112 and a radial direction 132 extending radially relative to the centre axis 112, can be defined. The medicament delivery device 1 comprises a housing 11 which has a proximal end 11a and a distal end 11b, and an outer cap 60 arranged proximally of the proximal end 11a of the housing 11.

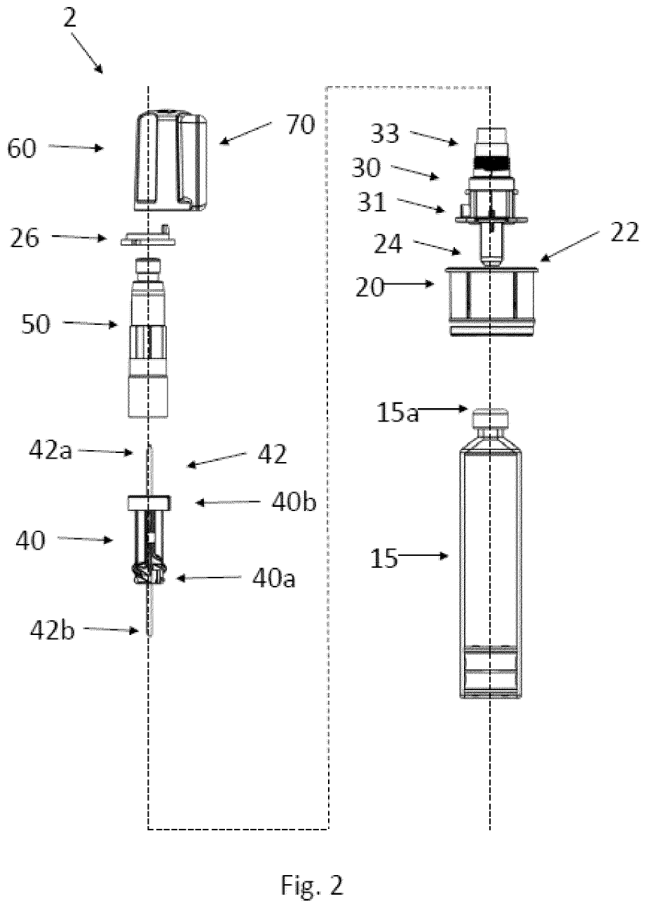

The medicament delivery device 1 further comprises a proximal housing part 20 removably attachable to the housing 11 of the medicament delivery device 1 at a proximal end 11a of the housing 11. The proximal housing part 20 comprises a proximal end portion 22 (better shown in FIG. 2). The outer cap 60 is operably attached to the proximal end portion 22 of the proximal housing part 20. As shown in FIGS. 1 and 2, the proximal end portion 22 may be annular. According to one alternative example embodiment, the proximal housing part 20 may be permanently attached to the housing 11, and e.g. form a part or portion of the housing 11.

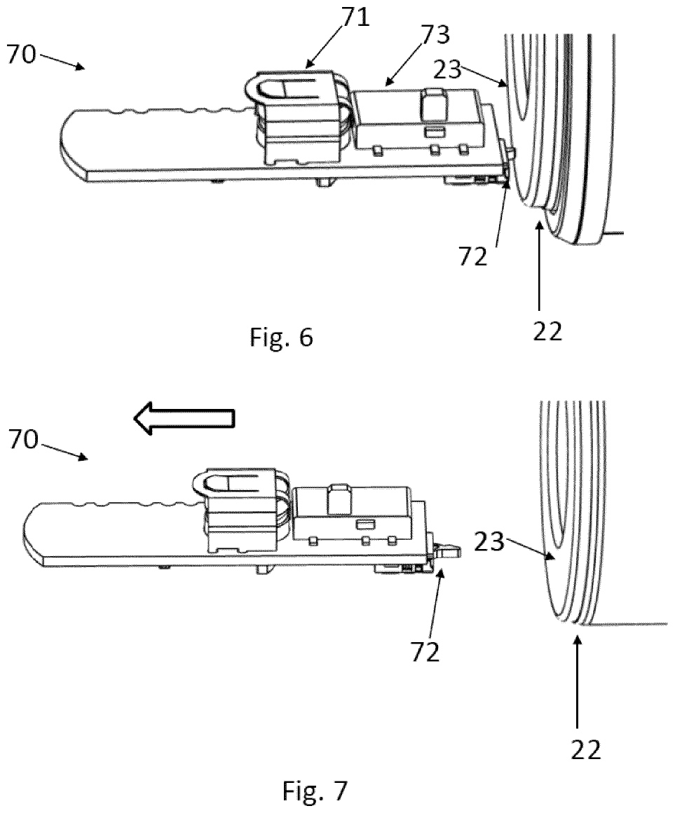

The medicament delivery device 1 further comprises a supplemental device 70 attached to the outer cap 60. The supplement device 70 comprises an activation member 72. The activation member 72 is configured to move from a non-activated state into an activated state when being moved from a fixed position relative to the proximal end portion 22 when the outer cap 60 is moved relative to the proximal housing part 20, which will be further explained in FIGS. 6-7.

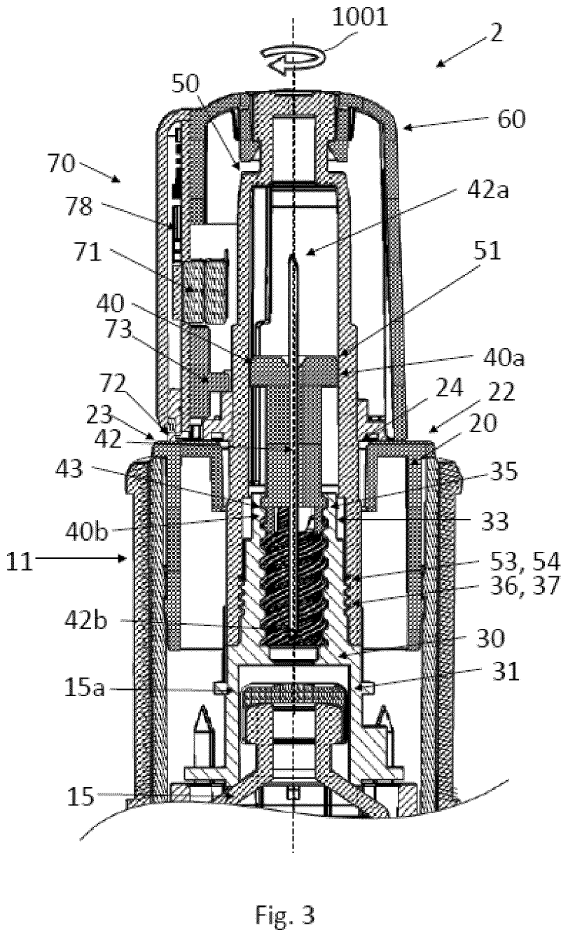

FIG. 2 is an exploded view of a subassembly 2 of the medicament delivery device 1. The subassembly 2 comprises the proximal housing part 20, the outer cap 60 and the supplement device 70 described with reference to FIG. 1. Moreover, the subassembly comprised a retainer 30 having a distal tubular portion 31 and a proximal tubular portion 33, better shown in FIG. 3. The retainer 30 is arranged at least partly inside of the proximal housing part 20.

The subassembly 2 further comprises a needle hub 40 extending from a proximal portion 40a to a distal portion 40b, and comprising a needle 42 having a proximal end 42a and an opposite distal end 42b.

The needle hub 40 and the needle 42 are axially movable within the retainer 30, which will further exemplified in FIG. 3.

The proximal end portion 22 of the proximal housing part 20 defines a proximal opening 24 through which the needle 42 extends in a proximal direction during a medicament delivery operation.

The subassembly 2 may further comprise a needle cap 50 attached to the outer cap 60 such that the needle cap 50 and the outer cap 60 are rotatable together. The outer cap 60 and the needle cap 50 may for example be arranged with interacting ribs, or interacting protrusions and indentations preventing relative rotational movement between the outer cap 60 and the needle cap 50. The needle cap 50 is operably attached to the needle hub 40 and the retainer, which will be further exemplified in FIG. 5.

The subassembly 2 may further comprise a cap clutch 26 proximally attached to the proximal end portion 22 of the proximal housing part 20, which will be further exemplified in FIG. 8.

In FIG. 2, the medicament container 15 is shown distally of the proximal housing part 20, the medicament container 15 has a proximal membrane 15a which seals the medicament inside the medicament container 15. The medicament container 15 may according to one embodiment form part of the subassembly 2.

The outer cap 60 is operably connected to the needle hub 40 such that when the outer cap 60 is manually operated, the needle hub 40 is distally and coaxially moved within the proximal tubular portion 33 of the retainer 30 whereby the distal end 42b of the needle 42 penetrates a proximal membrane 15a. This will be further exemplified in FIGS. 3-5.

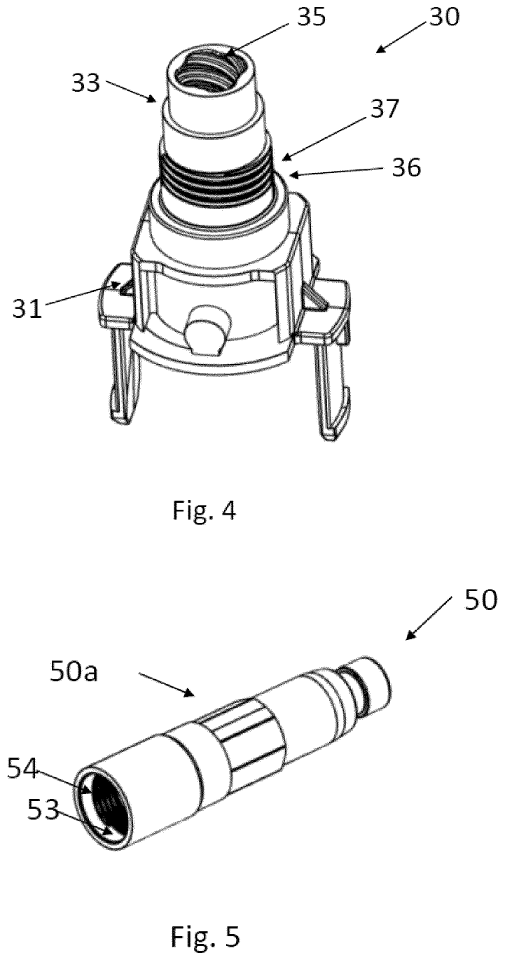

FIG. 3 is a detailed view of the subassembly 2 of FIG. 2 arranged inside of the housing 11 of the medicament delivery device 1 of FIG. 1, FIG. 4 is a perspective detailed view of the retainer 30 of FIGS. 2 and 3, FIG. 5 is a perspective detailed view of the needle cap 50 of FIGS. 2 and 3, and FIG. 6 is a perspective view of the supplement device 70 in relation to the proximal end portion 22 of the proximal housing part 20 of FIGS. 2 and 3.

As shown in FIGS. 3 and 4, the proximal tubular portion 33 of the retainer 30 comprises a first engaging structure 35. In the example embodiment of FIGS. 3 and 4, the first engaging structure 35 is an inner thread 35 arranged internally of the proximal tubular portion 33. The retainer 30 further comprises an outer surface 36 comprising a second engaging structure 37. In the example embodiment of FIGS. 3 and 4, the second engaging structure 37 is an outer thread 37 in the outer surface 36. Moreover, as shown in FIG. 3, the distal tubular portion 31 of the retainer 30 may be configured to house the proximal membrane 15a of the medicament container 15.

As shown in FIGS. 3 and 5, the needle cap 50 has a first inner surface 51 rotationally locked with the proximal portion 40a of the needle hub 40. This may e.g. be achieved by that a portion 50a of the needle cap 50 comprising the first inner surface 51 is non-circular, e.g. by being polygonally shaped. As a further alternative, the proximal portion 40a of the needle hub 40 and the inner surface 51 of the portion 50a of the needle cap 50 may be arranged with interacting ribs, or interacting protrusions and indentations preventing relative rotational movement between the needle cap 50 and the needle hub 40. Furthermore, the needle cap 50 has a second inner surface 53 comprising a third engaging structure 54 engageable with the second engaging structure 37 of the retainer 30. In the example embodiment of FIGS. 3 and 5, the third engaging structure 54 is an inner thread 54 in the second inner surface 53 of the needle cap 50. Thus, the third engaging structure 54 is threadedly engageable with the second engaging structure 37. However, it should be mentioned that instead of a threadedly engaging structure, the third engaging structure 54 may be arranged to engage with the second engaging structure 37 by a bayonet fitting, or a snap-fit.

As shown in FIG. 3, the distal portion 40b of the needle hub 40 comprises a fourth engaging structure 43 engageable with the first engaging structure 35 of the proximal tubular portion 33 of the retainer 30. In the example embodiment of FIG. 3, the fourth engaging structure 43 is an outer thread of the distal portion 40b of the needle hub 40. Thus, the fourth engaging structure 43 is threadedly engageable with the first engaging structure 35.

The outer cap 60 is configured to upon rotation in a first direction 1001, rotate the needle cap 50 such that the inner thread 54 of the needle cap 50 disengages from the outer thread 37 of the retainer 30 to enable the outer cap 60 and the needle cap 50 to be axially moved proximally away from the proximal housing part 20, i.e. to perform cap removal as described with references to FIGS. 7-8. Furthermore, upon rotation of the outer cap 60 in the first direction 1001, the outer thread 43 of the distal portion 40b of the needle hub 40 rotatably engages with the inner thread 35 of the proximal tubular portion 33 of the retainer 30 to distally move the needle hub 40 coaxially within the retainer 30. Hereby, the needle hub 40 is distally and coaxially moved within the proximal tubular portion 33 of the retainer 30 whereby the distal end 42b of the needle 42 can penetrate the proximal membrane 15a of the medicament container 15.

The supplemental device 70 is attached to the outer cap 60, and comprises besides the previously mentioned activation member 72, a battery 71, and a communication module 73 configured to transmit device status data to an external device upon activation of the activation member 72. Thus, the communication module 73 is configured to transmit device status data upon activation of the activation member 72. For example, the communication module 73 is configured to transmit the device status data wirelessly to the external device. The external device may e.g. be a smart device or a device of a network, such as a router. Possible wireless communication methods include Bluetooth and Cellular Networks.

The activation member 72 is configured to assume a non-activated state when being arranged in a fixed position relative to the proximal end portion 22 of the proximal housing part 20. As shown in FIG. 3, the proximal end portion 22 of the proximal housing part 20 comprises a proximal end surface 23. The fixed position of the activation member 72 is in the example embodiment of FIG. 3 defined by that the activation member 72 abuts the proximal end surface 23. This is also shown in FIG. 6. Thus, when the activation member 72 abuts the end surface 23 of the proximal end portion 22, the activation member 72 assume its non-activated state.

Turning to FIG. 7, showing the activation member 72 and the proximal end portion 22 of the proximal housing part 20 of FIG. 6, but where the activation member 72 has assumed an activated state by that the activation member 72 has been moved from the fixed position relative to the proximal end portion 22 and the proximal end surface 23 thereof. In more detail, the supplement device 70 has been axially and distally moved away from the proximal end portion 22 of the proximal housing part 20, indicated by the direction of the arrow in FIG. 7, such that the activation member 72 no longer abuts the proximal end surface 23 of the proximal end portion 22. Such movement of the supplement device 70 is carried out when the outer cap 60 is manually operated and is moved together with the supplement device 70 relative to the proximal housing part 20, typically by a cap removal action. In other words, the activation member 72 is configured to move to its activated state when the outer cap 60 is manually operated to be removed from the proximal housing part 20.

Activation of the activation member 72 by the cap removal action trigger transmission of status data from the communication module 73. As described with reference to FIG. 3, cap removal results in that the needle hub 40 is distally and coaxially moved within the proximal tubular portion 33 of the retainer 30 whereby the distal end 42b of the needle 42 can penetrate the proximal membrane 15a of the medicament container 15. Thus, the status data may e.g. comprise information of that the needle 42 has penetrated the proximal membrane 15b of the medicament container 15 and that the outer cap 60 is distant relative to the proximal housing part 20 (i.e. that the cap removal action has been performed).

The activation member 72 is preferably retractable. Thus, in the non-activated state shown in FIG. 6, the activation member 72 is arranged retracted by its interaction with the proximal end surface 23. In the activated state shown in FIG. 7, the activation member 72 is arranged protracted as it no longer abuts the proximal end surface 23. The activation member 72 may e.g. be retractable by means of a spring or other biasing means. That is, the activation member 72 may act as a mechanical switch.

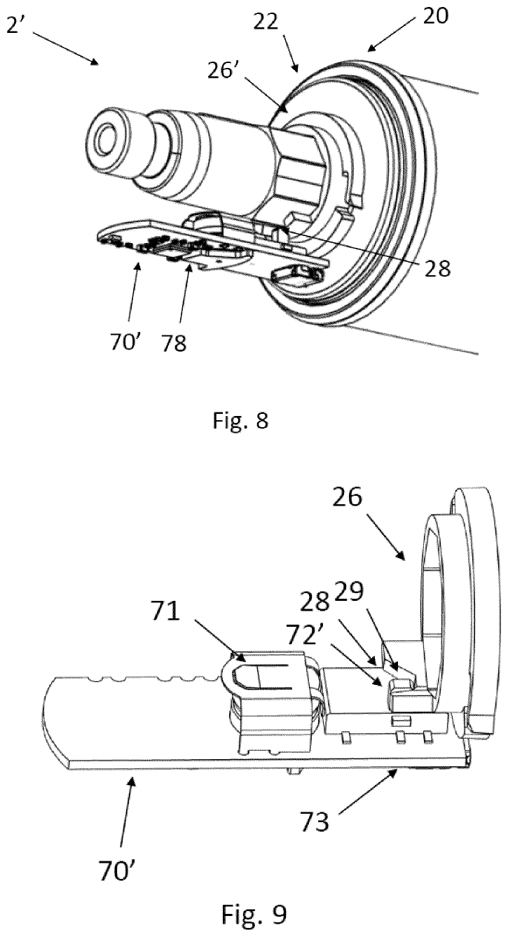

Turning to FIG. 8 showing an example embodiment of components of an alternative subassembly 2′ for which the outer cap 60 has been removed for increased visibility. The subassembly 2′is in large the same as the subassembly 2 in FIG. 3, why the same reference numerals are used for like features, and mainly the differences between the subassemblies 2, 2′ are discussed here. The differences between the subassemblies 2, 2′ are mainly related to the supplement device 70′ and the cap clutch 26′. In FIG. 8, the cap clutch 26′ is proximally attached to the proximal end portion 22 of the proximal housing part 20. As better shown in the detailed view of the cap clutch 26′ and the supplement device 70′ in FIG. 9, the cap clutch 26′ comprises a socket 28 with at least one activation ramp 29. In the example embodiment of FIG. 9, the socket 28 is wedged shaped and being defined by two mirrored activation ramps arranged in the circumferential direction 131. However, the socket 28 may have a different shape as long as it includes at least one activation ramp.

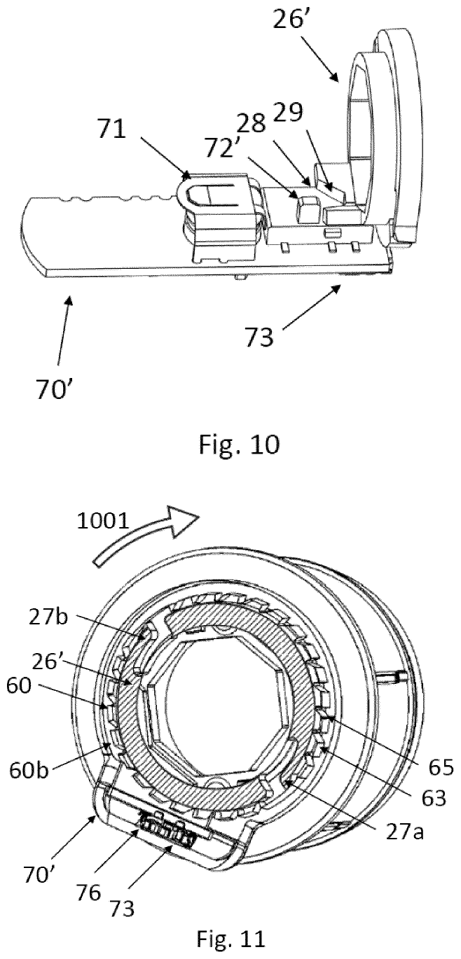

The supplement device 70′ comprises an activation member 72′. Correspondingly to the activation member 72 of the supplement device 70 of the subassembly 2 of FIG. 3, the activation member 72′ is configured to move from a non-activated state into an activated state when being moved from a fixed position relative to the proximal end portion 22 when the outer cap 60 is moved relative to the proximal housing part 20. However, in the subassembly 2′of FIG. 8, the fixed position of the activation member 72′ is defined by that the activation member 72′ is arranged within the socket 28 at a fixed axial distance from the proximal end portion 22 of the proximal housing part 20. The activation member 72′ is configured to move to its activated state by being moved out of the socket 28 via the activation ramp 29, as shown in FIG. 10. This is achieved by rotationally moving the outer cap 60 relative to the proximal housing part 20. That is, as the supplement device 70′ is attached to the outer cap 60, the outer cap 60 and the supplement device 70′ will move in unison, and a rotational movement of the outer cap 60 causes a rotational movement of the supplement device 70′, and hence the activation member 72′ which will be forced against the activation ramp 29. As the activation member 72′ abuts the activation ramp 29, the rotation movement of the supplement device 70′ will be at least partly translated into an axial movement of the activation member 72′, causing at least the activation member 72′ to move distally away from the socket 28 and thus, distally away from the proximal end portion 22 of the proximal housing part 20.

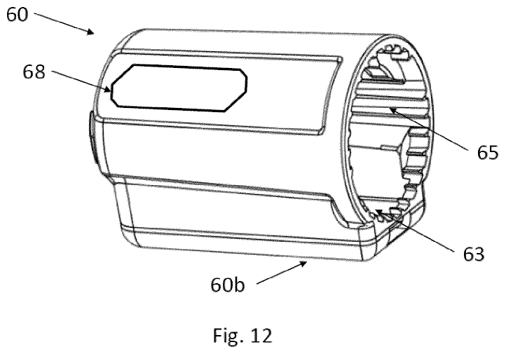

The rotational movement of the outer cap 60 of the subassembly 2′will now be described in relation to an example embodiment. As shown in the example embodiment of FIG. 11 and FIG. 12, the outer cap 60 may comprise a distal portion 60b with an inner surface 63 comprising axially and radially extending protrusions 65. The cap clutch 26′, shown in FIG. 11, comprises at least one radially flexibly arranged tongue 27a, in the example embodiment of FIG. 11 being a first radially flexibly arranged tongue 27a and a second radially flexibly arranged tongue 27b arranged opposite to the first radially flexibly arranged tongue 27a. The first and second radially flexibly arranged tongues 27a, 27b are arranged to interact with the axially and radially extending protrusions 65 of the inner surface 63 of the outer cap 60 by a ratchet mechanism. That is, the first and second radially flexibly arranged tongues 27a, 27b form a ratchet mechanism together with the axially and radially extending protrusions such that the first and second radially flexibly arranged tongues 27a, 27b engages with the axially radially extending protrusions 65 to rotate the cap clutch 26′ when the outer cap 60 is being rotated in a first rotational direction 1001. When the outer cap 60 is being rotated in an opposite direction to the first rotational direction 1001, the first and second radially flexibly arranged tongues 27a, 27b slides up and over the axially and radially extending protrusions 65 to generate a click sound. When the outer cap 60 is being rotated in an opposite direction to the first rotational direction 1001, the cap clutch 26′ is not rotated together with the outer cap 60. Thus, movement of the outer cap 60 in the first rotational direction 1001 may be referred to rotation of the outer cap 60 in the correct rotational direction, and movement of the outer cap 60 in the opposite direction to the first rotational direction 1001 may be referred to as rotation of the outer cap 60 in the wrong rotational direction.

The supplemental device 70′ may comprises a sensor 76 activable by the activation member 72′ and configured to identify the click sounds generated by the ratchet mechanism when the outer cap 60 is being rotated in the opposite direction to the first rotational direction 1001 (i.e. in the wrong rotational direction). For example, the sensor 76 may be an accelerometer.

The device status data transmitted from the communication module 73 may upon activation of the activation member 72′ comprise information of the rotational direction of the outer cap 60 based on the presence and absence of identification of click sounds by the sensor 76. For example, the device status data transmitted from the communication module 73 upon activation of the activation member 72′ may comprise information that the rotational direction of the outer cap 60 is wrong in response to identification of click sounds by the sensor 76.

According to one example embodiment, the outer cap 60 comprises light-emitting source 68, such as a LED-light, preferably integrated into an outer surface of the outer cap 60. The light-emitting source 68 may e.g. be powered by the battery 71 of the supplement device 70′. According to one example embodiment, the communication module 73 of the supplement device 70′ is configured to activate the light-emitting source 68 in response to receiving information that the rotational direction of the outer cap 60 is wrong. It is also conceivable that the user interface is something else than the light-emitting source 68 configured to provide feedback to the patient, such as haptic, and/or audio feedback.

The activation member 72, 72′ of the supplement device 70, 70′ may be a mechanical switch, i.e. a switch that is mechanically activated e.g. by interaction of an external force, as described with reference to FIGS. 6-7. However, the activation member 72, 72′ of the supplement device 70, 70′ may instead of a mechanical switch be a non-contact switch, such as a magnetic switch, a hall-effect sensor or a motion-based sensor. Moreover, the activation member 72, 72′ of the supplement device 70, 70′ may be, or comprise, an electronic switch.

Alternatively, or additionally, instead of a switch, the activation member comprises a first conductive end and a second conductive end. The first conductive end is operably connected to the battery, and the second conductive end is operably connected to the communication module. In this example, the proximal housing part comprises an insulating sheet. When the outer cap is adjacent to the proximal housing part, the insulating sheet is positioned between the first conductive end and the second conductive end such that the activation member is in non-activated state as the electrical connection between the battery and the communication module is ruptured by the insulating sheet. Once the outer cap is moved away from the proximal housing part, activation member is moved away from the insulating sheet such that the first conductive end is connected to the second conductive end. As a result, the electrical connection between the battery and the communication module is established and the activation member is in its activated state.

The supplemental device 70, 70′ may further comprises a memory 78 (only shown in the embodiment of FIG. 8) configured to store the status device data.

The subassembly 2, 2′ of the medicament delivery device 1 (such as an autoinjector) may generally include various other components. For example, a processing unit configured to control the entire system and processes the data before transmitting it.

Such processing units may comprise a logic circuit or control unit including a microprocessor, microcontroller, programmable digital signal processor or another programmable device. The processing circuitry may also, or instead, each include an application specific integrated circuit, a programmable gate array or programmable array logic, a programmable logic device, or a digital signal processor. Where the processing circuitry includes a programmable device such as the microprocessor, microcontroller or programmable digital signal processor mentioned above, the processor may further include computer executable code that controls operation of the programmable device.

Moreover, the subassembly 2, 2′ of the medicament delivery device 1 may comprise a secondary sensor unit which may recognize injection events, such as the autoinjector inserted into an attachment portion of e.g., a pad, injection started, and injection ends. Here, the memory 78 of the supplement device 70, 70′ may be configured to store the recorded data during the injection, the communication module 73 may be configured to transmit the stored data to the external device.

As previously mentioned, the communication module 73 may be operated by wireless communication methods such as Bluetooth and Cellular Networks. Bluetooth connectivity requires a smart device to transmit the stored data to the network and it requires a pairing action between the pad and the smart device before being able to use the supporting pad in case of 2-way connection. But it's a cheaper alternative and it requires less space on the PCB. A 1-way connection does not require pairing. The cellular network does not require any pairing process, it can be used as a plug-n-play device, no prior setup is needed, but it's more expensive and it requires more space on PCB. Depending on the requirements of the product any of those two technologies can be used.

The medicament delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary types of drugs that could be included in the medicament delivery devices described herein include, but are not limited to, antibodies, proteins, fusion proteins, peptibodies, polypeptides, pegylated proteins, protein fragments, protein analogues, protein variants, protein precursors, and/or protein derivatives. Exemplary drugs that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-1a (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatitis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)). Pharmaceutical formulations including, but not limited to, any drug described herein are also contemplated for use in the medicament delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

The inventive concept has mainly been described above with reference to a few examples. However, as is readily appreciated by a person skilled in the art, other embodiments than the ones disclosed above are equally possible within the scope of the inventive concept, as defined by the appended claims. For example, the outer cap may be operably connected to the needle hub and to the retainer by other means than those presented here, as long as the needle hub is distally moved such that the distal end of the needle penetrates the membrane when the outer cap is removed and detached from the retainer.

Furthermore, in another embodiment, the supplement device 70″, 70″ can be used in a medicament delivery device that is configured to expel medicament from a syringe. In other words, the medicament delivery device comprises a medicament container that has a barrel for containing medicament and a needle integral at the proximal end of the barrel. In this example, the needle hub and the retainer as mentioned above are not necessary.

In this example, the needle is configured to be sealed by a flexible needle shield or a flexible needle shield that is surrounded by a rigid needle shield. The needle shield is configured to be removed when the outer cap 60′, 60″ is removed from the housing 11. In this embodiment, the outer cap 60′, 60″ comprises a needle shield remover configured to be engaged with the flexible needle shield and/or the rigid needle shield so that the needle shield can be removed once the outer cap 60′, 60″ is removed from the housing 11. The needle shield remover can be an integral part of the outer cap 60′, 60″ or an independent component that is attached to the outer cap 60′, 60″. In this embodiment, the cap 60′, 60″ is configured to be removed from the housing 11 via an initial rotation around the axis 112 relative to the housing 11 that is performed by a user. It should be noted that the initial rotation around the axis 112 can result in the outer cap 60′; 60″ being moved in the circumferential direction with any rotational degrees dependent on the design. For example, the outer cap can be rotated with 30 degrees around the axis 112 relative to the housing 11 as the initial rotation. After the initial rotational movement, the outer cap can be moved further away from the housing 11 by the user with a linear movement along the axis 112 or a combination of rotation and axial movement. Furthermore, the initial rotational movement of the outer cap is a combination of movement including the initial rotation and an axial movement along the axis 112. In one example, the activation member of the supplement device is configured to be in contact with the proximal end of the housing 11 or a proximal end portion to state in the non-activated state as mentioned above.

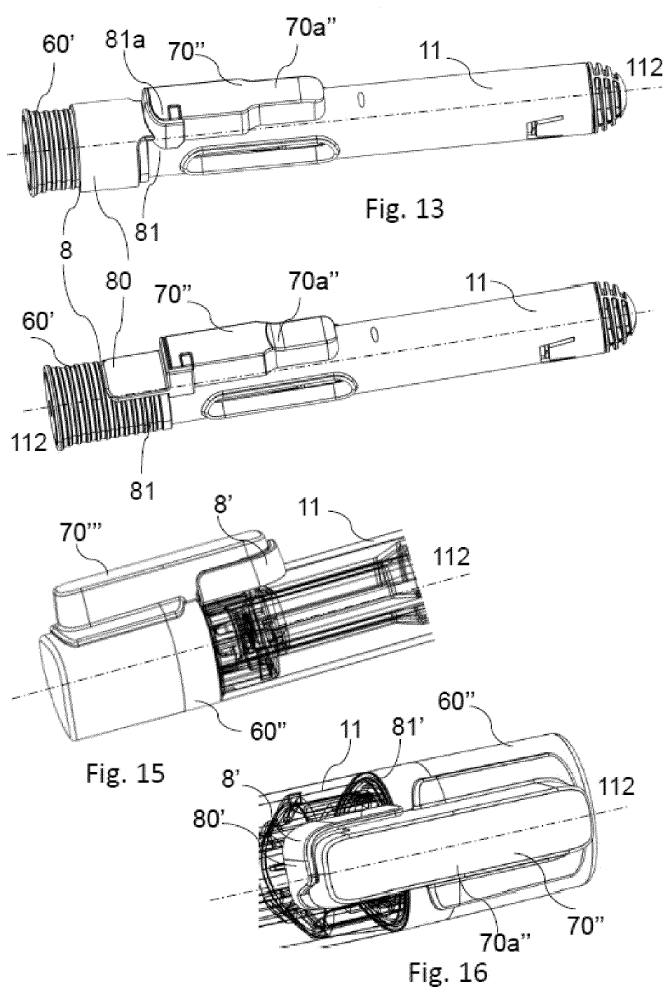

Alternatively, the medicament delivery device comprises an adapter 8, 8′ as shown in FIGS. 13-16. In this example, the adapter 8, 8′ is configured to interact with the supplement device 70″, 70′″ to guide the outer cap 60′, 60″ to be rotated around the axis 112. In one example, the supplement device 70″, is attached to the housing 11 and the adapter 8 is attached to the outer cap 60′, as shown in FIGS. 13-14. Alternatively, the supplement device 70′″ is attached to the outer cap 60″ and the adapter 8′is attached to the housing 11, as shown in FIGS. 15-16.

In one example, the adapter comprises protrusion 81; 81′configured to be engaged with a surface of the supplement device 70″; 70′″ that faces in the radial direction 132. In a preferred example, the supplement device 70″; 70′″ comprises a shell 70a″, 70a′″, in this example, the surface of the supplement device 70″; 70′″ is a side surface of the shell 70a″, 70a′″. In a preferred example, the shell 70a″ comprises a recess/cut-out facing in the radial direction 132. In this example, the adapter 8 comprises a rib 81a′ configured to be placed into the recess/cut-out, as shown in FIG. 13. Therefore, the outer cap 60′ cannot be removed from the housing 11 without an initial rotation around the axis 112.

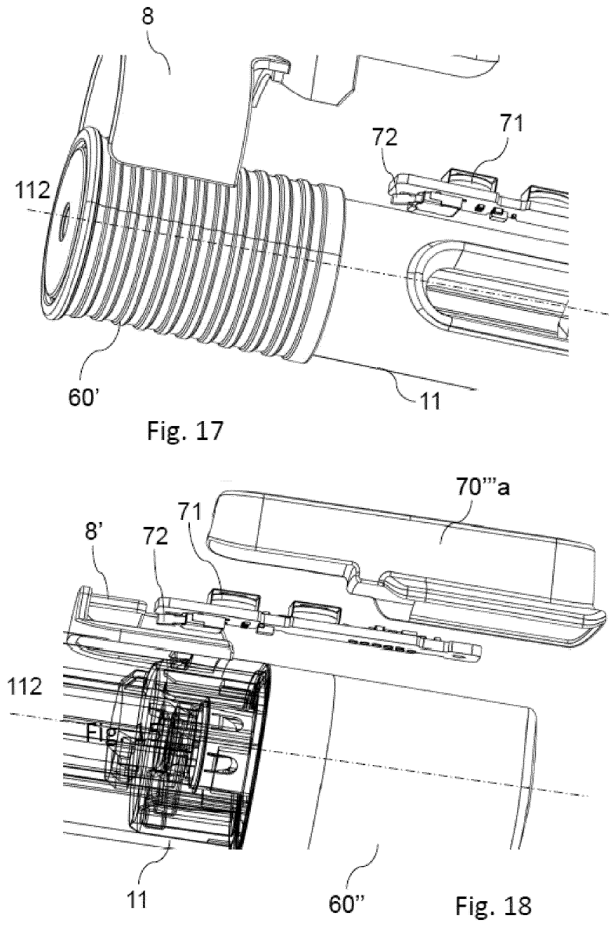

In one example where the medicament delivery device comprises the adapter 8, 8′, the adapter 8, 8′ comprises a distally directed surface configured to press on a proximally directed surface of the activation member 72, as shown in FIGS. 17-18. In this example, the activation member 72 can be a mechanical switch. The mechanical switch is movable by a built-in resilient member so that when the mechanical switch is pressed, the activation member 72 is in its non-activated state. Once the mechanical switch is no longer pressed, the built-in resilient member is configured to move the mechanical switch to its activated state. Alternatively, the activation member 72 can be a non-contact switch. In this example, the adapter does not need to be in contact with the activation member 72. For example, the activation member 72 can be a reed switch; in this example, the adapter comprises a magnet.

Some aspects of the invention are defined by the clauses below.
1. A subassembly (2, 2′) of a medicament delivery device (1) configured to expel medicament from a medicament container (15) having a proximal membrane (15a), the subassembly (2, 2′) comprising:
   a retainer (30) having a distal tubular portion (31) and a proximal tubular portion (33),
   a needle hub (40) comprising a needle (42) having a proximal end (42a) and an opposite distal end (42b), the needle hub (40) extending from a proximal portion (40a) to a distal portion (40b),
   a proximal housing part (20) having a proximal end portion (22) defining a proximal opening (24) through which the needle (42) extends in a proximal direction during a medicament delivery operation,
   an outer cap (60) operably attached to the proximal end portion (22) of the proximal housing part (20), wherein the outer cap (60) is operably connected to the needle hub (40) such that when the outer cap (60) is manually operated, the needle hub (40) is distally and coaxially moved within the proximal tubular portion (33) of the retainer (30) whereby the distal end (42b) of the needle (42) penetrates the proximal membrane (15a),
   a supplemental device (70, 70′) attached to the outer cap (60), the supplemental device (70, 70′) comprising a battery (71), an activation member (72, 72′) and a communication module (73) configured to transmit device status data to an external device upon activation of the activation member (72, 72′), wherein the activation member (72, 72′) is configured to assume a non-activated state when being arranged in a fixed position relative to the proximal end portion (22) of the proximal housing part (20),
   wherein the activation member (72, 72′) is configured to move from its non-activated state into an activated state when being moved from the fixed position relative to the proximal end portion (22) when the outer cap (60) is moved relative to the proximal housing part.
2. The subassembly (2) according to clause 1, wherein the communication module (73) is configured to transmit device status data upon activation of the activation member (72, 72′).
3. The subassembly (2) according to any one of clauses 1-2, wherein the proximal end portion (22) of the proximal housing part (20) comprises a proximal end surface (23), and wherein the fixed position of the activation member (72) is defined by that the activation member (72) abuts the proximal end surface (23).
4. The subassembly (2) according to clause 3, wherein the activation member (72) is configured to move to its activated state when the outer cap (60) is manually operated to be removed from the proximal housing part (20).
5. The subassembly (2) according to any one of clauses 4-5, wherein the device status data transmitted from the communication module (73) upon activation of the activation member (72) comprises information of that the needle (42) has penetrated the proximal membrane (15b) of the medicament container (15) and the outer cap (60) is distant relative to the proximal housing part (20).
6. The subassembly (2′) according to any one of clauses 1-2, further comprising a cap clutch (26′) proximally attached to the proximal end portion (22) of the proximal housing part (20), the cap clutch (26′) comprising a socket (28) with at least one activation ramp (29), and wherein the fixed position of the activation member (72′) is defined by that the activation member (72′) is arranged within the socket (28) at a fixed axial distance from the proximal end portion (22).
7. The subassembly (2′) according to clause 6, wherein the activation member (72′) is configured to move to its activated state by moving the activation member (72′) out of the socket (28) via the activation ramp (29), when the outer cap (60) is rotationally moved relative to the proximal housing part (20).
8. The subassembly (2′) according to clause 7, wherein the outer cap (60) comprises a distal portion (60b) with an inner surface (63) comprising axially and radially extending protrusions (65), and wherein the cap clutch (26′) comprises at least one radially flexibly arranged tongue (27a, 27b) arranged to interact with the axially and radially extending protrusions (65) by a ratchet mechanism, such that the radially flexibly arranged tongue (27a, 27b) engages with the axially radially extending protrusions (65) to rotate the cap clutch (26′) when the outer cap (60) is being rotated in a first rotational direction (1001), and such that the radially flexibly arranged tongue (27a, 27b) slides up and over the axially and radially extending protrusions (65) to generate a click sound when the outer cap (60) is being rotated in an opposite direction to the first rotational direction (1001).
9. The subassembly (2′) according to clause 8, wherein the supplemental device (70′) further comprises a sensor (76) activable by the activation member (72′) and configured to identify the click sounds generated by the ratchet mechanism when the outer cap (60) is being rotated in the opposite direction to the first rotational direction (1001).
10. The subassembly (2) according to clause 9, wherein the sensor (76) is an accelerometer.
11. The subassembly (2) according to any one of clauses 9-10, wherein the device status data transmitted from the communication module (73) upon activation of the activation member (72′) comprises information of the rotational direction of the outer cap (60) based on the presence and absence of identification of click sounds by the sensor (76).
12. The subassembly (2′) according to any one of clauses 10-11, wherein the device status data transmitted from the communication module (73) upon activation of the activation member (72′) comprises information that the rotational direction of the outer cap (60) is wrong in response to identification of click sounds by the sensor (76).
13. The subassembly (2) according to clause 12, further comprising a light-emitting source, wherein the communication module (73) is configured to activate the light-emitting source in response to receiving information that the rotational direction of the outer cap (60) is wrong.
14. The subassembly (2) according to any one of the preceding clauses, wherein the activation member (72) is retractable, and is arranged retracted in its non-activated state, and is arranged protracted in its activated state.
15. The subassembly (2, 2′) according to any one of the preceding clauses, wherein the activation member (72, 72′) is an electronic switch.
16. The subassembly (2) according to any one of the preceding clauses, wherein the supplemental device (70, 70′) further comprises a memory (78) configured to store the status device data, and wherein the communication module (73) is configured to transmit the device status data wirelessly to the external device.
17. The subassembly (2, 2′) according to any one of the preceding clauses, wherein the proximal tubular portion (33) of the retainer (30) comprises a first engaging structure (35), and wherein the retainer (30) comprises an outer surface (36) comprising a second engaging structure (37).
18. The subassembly (2, 2′) according to clause 17, further comprising a needle cap (50) having a first inner surface (51) rotationally locked with the proximal portion (40a) of the needle hub (40), and a second inner surface (53) comprising a third engaging structure (54) engageable with the second engaging structure (37) of the retainer (30), and where the distal portion (40b) of the needle hub (40) comprises a fourth engaging structure (43) engageable with the first engaging structure (35) of the proximal tubular portion (33) of the retainer (30).
19. The subassembly (2, 2′) according to clauses 17-18, wherein the first engaging structure (35) is an inner thread (35) in the proximal tubular portion (33), the second engaging structure (37) is an outer thread (37) in the outer surface (36), the third engaging structure (54) is an inner thread (54) in the second inner surface (53) of the needle cap (50), and the fourth engaging structure (43) is an outer thread of the distal portion (40b) of the needle hub (40), and wherein the outer cap (60) is configured to upon rotation in the first direction (1001), rotate the needle cap (50) such that the inner thread (54) of the needle cap (50) disengages from the outer thread (37) of the retainer (30) to enable the outer cap (60) and the needle cap (50) to be axially moved proximally away from the proximal housing part (20), and such that the outer thread (43) of the distal portion (40b) of the needle hub (40) rotatably engages with the inner thread (35) of the proximal tubular portion (33) of the retainer (30) to distally move the needle hub (40) coaxially within the retainer (30).
20. The subassembly (2, 2′) according to any one of the preceding clauses, wherein the distal tubular portion (31) of the retainer (30) is configured to house the proximal membrane (15a) of the medicament container (15).
21. A medicament delivery device (1) configured to expel medicament from a medicament container (15), the medicament delivery device (1) comprising a sub-assembly (2, 2′) according to any one of clauses 1-20.
22. A medicament delivery device comprises a housing (11), an outer cap (60′, 60″), and a supplement device (70″, 70″); wherein the housing (11) extends between a proximal end and a distal end along an axis (11); wherein the outer cap (60′, 60″) is attached to the proximal end of the housing (11) and is configured to be removed from the housing (11) by an initial rotation around the axis (112) relative to the housing (11); wherein the supplement device (70″, 70″) comprises an activation member (72); wherein the activation member (72) is configured to move from its non-activated state into an activated state when the outer cap (60) removed from the housing (11).
23. The medicament delivery device according to clause 22, wherein the medicament delivery device comprises an adapter (8, 8′) configured to interact with the supplement device (70″, 70″) so that the outer cap (60′, 60″) can be guided to be rotated round the axis (112).
24. The medicament delivery device according to clause 23, wherein the adapter (8) is configured to prevent the outer cap (60′) to be removed from the housing (11) without the initial rotation around the axis (112).
25. The medicament delivery device according to clauses 22-24, wherein the supplement device (70″) is attached to the housing.
26. The medicament delivery device according to clauses 22-24, wherein the supplement device (70″) is attached to the outer cap (60″).

1-15. (canceled)

16. A subassembly of a medicament delivery device configured to expel medicament from a medicament container having a proximal membrane, the subassembly comprising
a retainer having a distal tubular portion and a proximal tubular portion;
a needle hub comprising a needle having a proximal end and an opposite distal end, the needle hub extending from a proximal portion to a distal portion;
a proximal housing part having a proximal end portion defining a proximal opening through which the needle extends in a proximal direction during a medicament delivery operation;
an outer cap operably attached to the proximal end portion of the proximal housing part, wherein the outer cap is operably connected to the needle hub such that when the outer cap is manually operated, the needle hub is distally and coaxially moved within the proximal tubular portion of the retainer whereby the distal end of the needle penetrates the proximal membrane; and
a supplemental device attached to the outer cap, the supplemental device comprising a battery, an activation member and a communication module configured to transmit device status data to an external device upon activation of the activation member, wherein the activation member is configured to assume a non-activated state when being arranged in a fixed position relative to the proximal end portion of the proximal housing part,
wherein the activation member is configured to move from its non-activated state into an activated state when being moved from the fixed position relative to the proximal end portion when the outer cap is moved relative to the proximal housing part.

17. The subassembly according to claim 16, wherein the communication module is configured to transmit device status data upon activation of the activation member.

18. The subassembly according to claim 16, wherein the proximal end portion of the proximal housing part comprises a proximal end surface, and wherein the fixed position of the activation member is defined by that the activation member abuts the proximal end surface.

19. The subassembly according to claim 18, wherein the activation member is configured to move to its activated state when the outer cap is manually operated to be removed from the proximal housing part.

20. The subassembly according to claim 19, wherein the device status data transmitted from the communication module upon activation of the activation member comprises information of that the needle has penetrated the proximal membrane of the medicament container and the outer cap is distant relative to the proximal housing part.

21. The subassembly according to claim 16, further comprising a cap clutch proximally attached to the proximal end portion of the proximal housing part, the cap clutch comprising a socket with at least one activation ramp, and wherein the fixed position of the activation member is defined by that the activation member is arranged within the socket at a fixed axial distance from the proximal end portion.

22. The subassembly according to claim 21, wherein the activation member is configured to move to its activated state by moving the activation member out of the socket via the activation ramp, when the outer cap is rotationally moved relative to the proximal housing part.

23. The subassembly according to claim 22, wherein the outer cap comprises a distal portion with an inner surface comprising axially and radially extending protrusions, and wherein the cap clutch comprises at least one radially flexibly arranged tongue arranged to interact with the axially and radially extending protrusions by a ratchet mechanism, such that the radially flexibly arranged tongue engages with the axially radially extending protrusions to rotate the cap clutch when the outer cap is being rotated in a first rotational direction, and such that the radially flexibly arranged tongue slides up and over the axially and radially extending protrusions to generate a click sound when the outer cap is being rotated in an opposite direction to the first rotational direction.

24. The subassembly according to claim 23, wherein the supplemental device further comprises a sensor activable by the activation member and configured to identify the click sounds generated by the ratchet mechanism when the outer cap is being rotated in the opposite direction to the first rotational direction.

25. The subassembly according to claim 24, wherein the sensor is an accelerometer.

26. The subassembly according to claim 24, wherein the device status data transmitted from the communication module upon activation of the activation member comprises information of the rotational direction of the outer cap based on the presence and absence of identification of click sounds by the sensor.

27. The subassembly according to claim 25, wherein the device status data transmitted from the communication module upon activation of the activation member comprises information that the rotational direction of the outer cap is wrong in response to identification of click sounds by the sensor.

28. The subassembly according to claim 27, further comprising a light-emitting source, wherein the communication module is configured to activate the light-emitting source in response to receiving information that the rotational direction of the outer cap is wrong.

29. The subassembly according to claim 16, wherein the activation member is an electronic switch.

30. The subassembly according to claim 16, wherein the activation member comprises a first conductive end and a second conductive end, wherein the first conductive end is operably connected to the battery, and the second conductive end is operably connected to the communication module, and wherein the proximal housing part comprises an insulating sheet positioned between the first conductive end and the second conductive end such that the activation member is in non-activated state when the activation member is in its fixed position.
